(19) United States
(12) Patent Application Publication
(10) Pub. No.: US 2018/0303764 A1
(43) Pub. Date: Oct. 25, 2018

(54) METHOD FOR MANUFACTURING A TRANSDERMAL DEVICE

(71) Applicant: INEP EUROPE SARL, Luxembourg (LU)

(72) Inventors: SPIROS FOTINOS, ATTIKA (GR); JEROME LANGLUME, PARIS (FR); JEAN PAUL CARAVITA, PARIS (FR)

(21) Appl. No.: 15/838,837

(22) Filed: Dec. 12, 2017

Related U.S. Application Data
(63) Continuation-in-part of application No. 15/491,419, filed on Apr. 19, 2017.
Application No. 15/491,419 is a continuation-in-part of application No. 15/492,146, filed on Apr. 20, 2017.

(51) Int. Cl. A61K 9/70; A61F 13/02

(52) U.S. Cl. CPC A61K 9/7038; A61F 13/0283; A61F 2013/0296; A61F 13/0289; A61K 9/7084

(57) ABSTRACT

The invention relates to a method for manufacturing a transdermal patch from a drug-containing web that minimizes waste. The web is a layered composite that includes at least a backing layer and a drug-in-adhesive layer and a first strippable release liner. The web is kiss-cut along intersecting cut lines at least down to the depth of the liner, generally defining the extent of individual transdermal patches. The intersections of the cut lines define small zones that are punched out of the web in a generally star shape. The portions of the web above the liner are peeled away from the liner and transferred to a faster moving second liner so that the patches are now further spaced apart from one another. This second liner is then cut to provide transdermal patches that are mounted to release liners that are substantially broader in extent than the patches themselves.

### TECHNICAL FIELD OF THE INVENTION

The present invention is directed to the field of transdermal patches, and specifically to a method for manufacturing a transdermal patch.

### BACKGROUND OF THE INVENTION

Transdermal patches are drug delivery systems that typically include a backing material, a drug and an adhesive to adhere the patch to skin. Conventional uses include delivering a broad assortment of therapeutic agents, including nicotine and pain medications.

A typical conventional patch is shown in FIG. 1. Transdermal patch 100 includes backing layer 110 and drug-in-adhesive layer 120, may optionally also include rate controlling membrane 130 and adhesive layer 140 and other layers, as well. Regardless of the particular layering employed, these patches are typically sealed into pouches. To prevent the drug-in-adhesive layer 120 or the adhesive layer 140 from adhering to the pouch, the exposed drug-in-adhesive layer 120 or the exposed adhesive layer 140 of transdermal patch 100, as the case may be, is typically attached to an additional layer added, as illustrated in FIG. 2. The additional layer is strippable release liner 150. Release liner 150 prevents adherence of the exposed adhesive to surfaces during processing, prevents adherence to the primary package and allows patient handling of the patch prior to application to the patient's skin. The surface of release liner 150 attached to the adhesive has a special surface layer designed to release cleanly from the adhesive so that release liner 150 can easily be peeled from the adhesive by the patient without damage to the adhesive.

The conventional process for producing the prior art patches includes providing a spool of web material 170 that is made up of such subsidiary layers as are desired. Web material moves along a conveyor to a cutting station, where generally rectangular cuts 160 are made to form transdermal patches 100 as shown in FIG. 3. These cuts are often referred to as “kiss cuts”, in that unlike through-cuts, they extend only partially through the depth of the web material 170, typically through all the layers except for strippable release liner 150 as shown in FIG. 4. Rectangular zones between transdermal patches 100 are spaced a substantial distance from one another as shown, and define a waste 175 as shown in FIG. 5 material that, when removed, leaves behind a series of spaced apart rectangular islands that become transdermal patches 100 as placed on skin as shown in FIG. 4.

As shown in FIG. 4, the remaining web is then through-cut by vertical and horizontal lines 180 and then separated out into individual transdermal patches 100, each mounted to its supporting release liner 150 as is shown in FIG. 6. FIG. 6 shows a typical such arrangement of transdermal patch 100 (its specific layers not shown) mated to a generally rectangular strippable release liner 150.

The substantial waste material 175 that this process produces and then discards is very wasteful of the therapeutic agents that are at the heart of the transdermal patch.

A substantial fraction of the cost of a transdermal patch lies in the cost of the drug that it delivers. Known methods for manufacturing these patches begin with a continuous web that is a composite of the layers noted above, including the layer that carries the therapeutic agent, and the release liner film covering the exposed adhesive. The continuous web has indeterminate length, typically more than one thousand meters, and can have a width up to about one meter. This continuous web is converted to individual patches, the finished product, by punching the patch from the continuous web to the required size and shape and individually packaging the punched finished dosage, the patch, into a pouch. The longest dimension of the patch is much less than the width of the web so that many individual units are punched across the width. Punching of patches from this continuous web often results in significant losses of the drug due to spaces that may be required between the punched areas of the web. Because the layer that contains the drug is continuous, and because of losses of this layer in the process for punching the continuous web into individual dosages, an inefficient punching process can substantially increase production costs.

There remains a need for a method of manufacturing transdermal patches that more efficiently utilizes the therapeutic agents contained within the starting continuous web.

### SUMMARY OF THE INVENTION

The present invention relates to a method of manufacturing a transdermal device from a continuous layered web which is conveyed linearly. Upper layers of the web can be kiss-cut along both horizontal lines, lengthwise down the web, and vertical lines, crosswise across the web. The lines can be cut down from a backing side of the transdermal device to the depth of the underlying strippable release liner, leaving the strippable release liner uncut and intact. Alternatively, the horizontal and vertical cuts can be through-cuts, which cut through all layers of the web. Several patterns of kiss-cuts and through-cuts, applied to the continuous web, are described.

Individual portions of the web are then peeled away from the liner of the starting web by machinery with a sharp edge that raises the front rim of the forward moving portion. For example, the individual portions can be a patch being generally rectangular in shape with rounded corners as defined by a punch employed to stamp the individual portions. The front rim of the individual portions then contacts a second web of strippable release liner that moves faster than the first web, resulting in transfer of the entire individual portions to the second web. As a continuous process applied to a line of contiguous patches, this results in a lateral spacing apart of the transdermal patches on this second web along the direction of movement of the second web. The patches in the second web may be through-cut without transfer to a new release liner yielding a finished product with the strippable release liner contiguous with the patch. While this process uses more release liner material, it conserves use of the more expensive drug bearing material. The rounded corners of the patch are cut during the process, as kiss-cuts, and the drug material filling the rounded corners is removed as waste. The rounded corners can be cut before or after the horizontal through-cuts down the length of the web.

In some cases, the vertical kiss-cut does not result in a clean separation of the contiguous patches, because the adjacent adhesive surfaces along the kiss-cut re-adhere after the kiss-cut. Each of lines can include a pair of lines leaving a thin strip of waste material between the patches. The thin strip is removed with the rounded corners leaving a narrow space between contiguous patches, preventing re-adherence between the contiguous patches. This process yields slightly more waste than the process with the single kiss-cut between the patches, but substantially reduces waste relative to the conventional process.

The process of the present invention provides a transdermal patch formed by efficient use of the drug-in-adhesive or other drug-carrying layer, thereby reducing waste of the most expensive portion of the transdermal patch.

The invention will be more fully described by reference to the following drawings.

### BRIEF DESCRIPTION OF THE DRAWINGS

FIG. 1 is a cross-sectional view of a prior art web of starting material.

FIG. 2 is a cross-sectional view of a web of starting material from which the patch is formed, with a strippable release liner attached to the prior art patch.

FIG. 3 is a top plan view of the prior art web of starting material with rectangular sections defined by kiss-cuts shown in dashed lines.

FIG. 4 is a perspective view of the prior art web of FIG. 3, after removal of the waste material between the rectangular sections.

FIG. 5 is a top plan view of the prior art waste, with continuous lines showing where the rectangular sections have been removed;

FIG. 6 is a perspective view of a prior art transdermal patch.

FIG. 7 is a top plan view of a layered web as is utilized by the method of the present invention.

FIG. 8 depicts the web of FIG. 7, after it has been cross-cut and star-shaped sections removed at the corners. The dashed-line cross-cuts represent kiss-cuts and the continuous vertical lines represent through-cuts.

FIG. 9A is a top plan view of one strip of the layered web after it has been removed from the continuous web.

FIG. 9B is a cross-section of the strip of layered web shown in FIG. 9A.

FIG. 10 is a schematic illustration of the process of removing sections of the web from a first strippable release liner and transferring the sections to a second liner on a second line. The speed of the second web (V2) is greater than the speed of the first web (V1).

FIG. 11 is a top plan view of a finished transdermal patch produced by the method of the present invention.

FIG. 12 is a top plan view of a finished transdermal patch produced by the method of the present invention.

FIG. 13 depicts the web of FIG. 8, in which horizontal lines and vertical lines include a pair of horizontal lines and vertical lines leaving a thin strip of waste material between the patches.

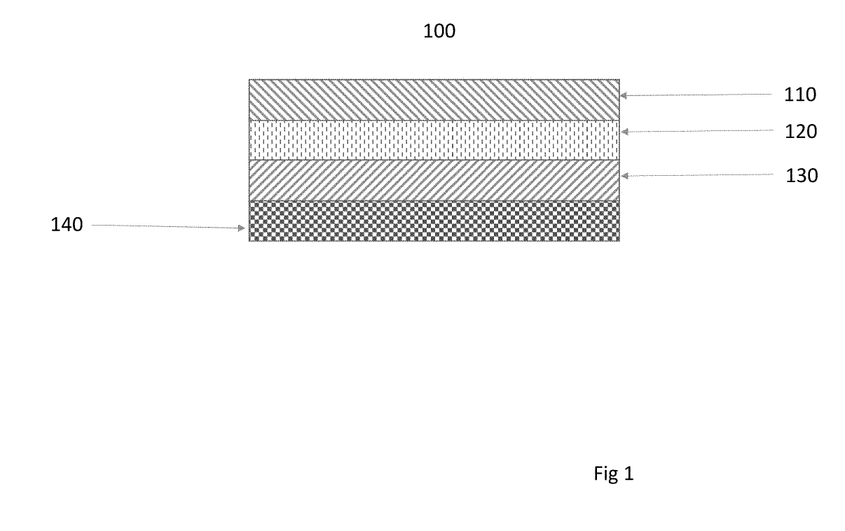

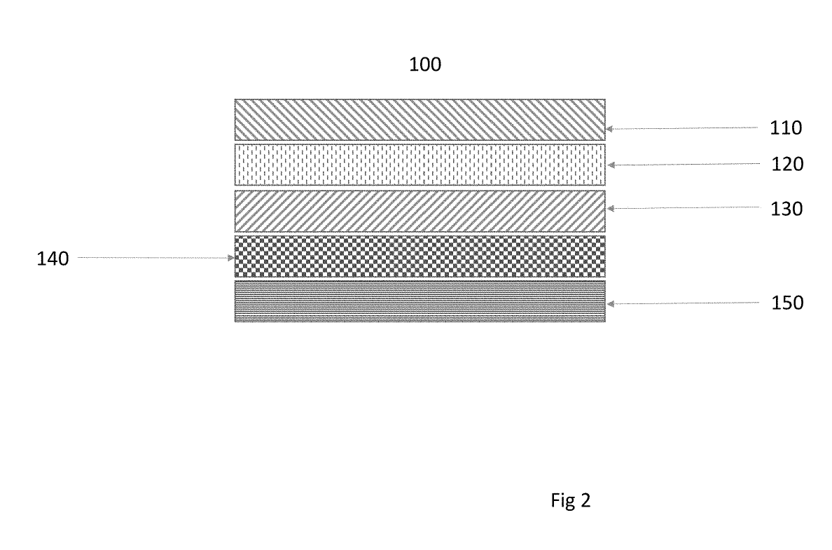

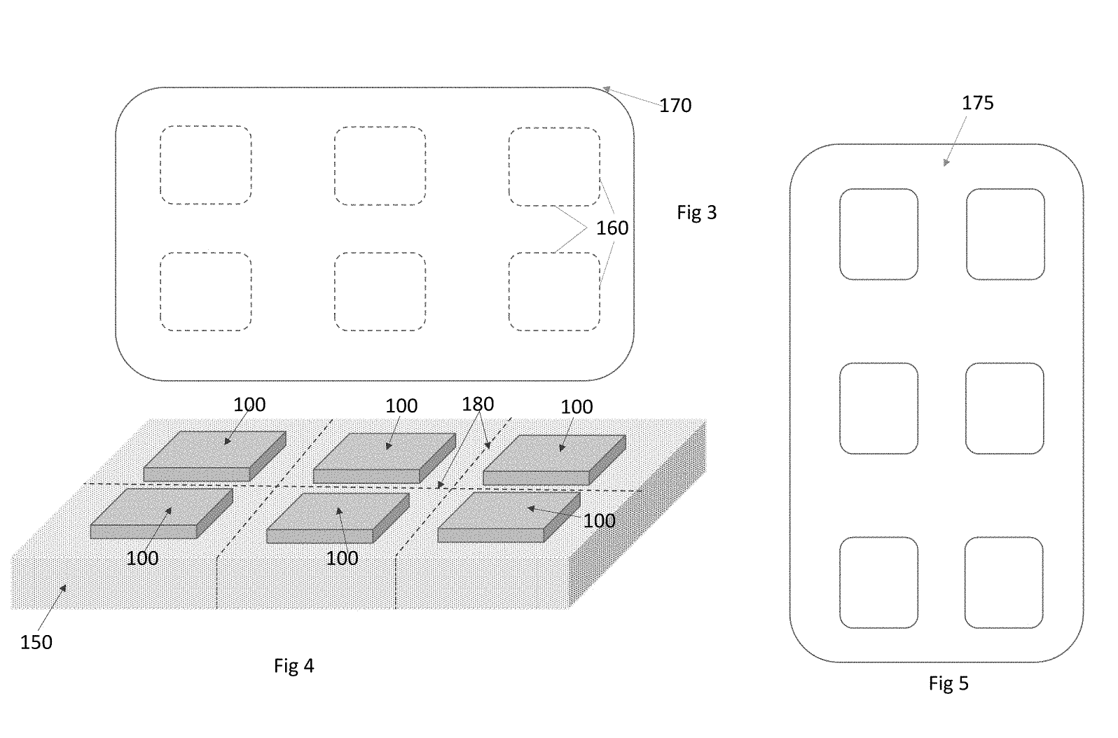

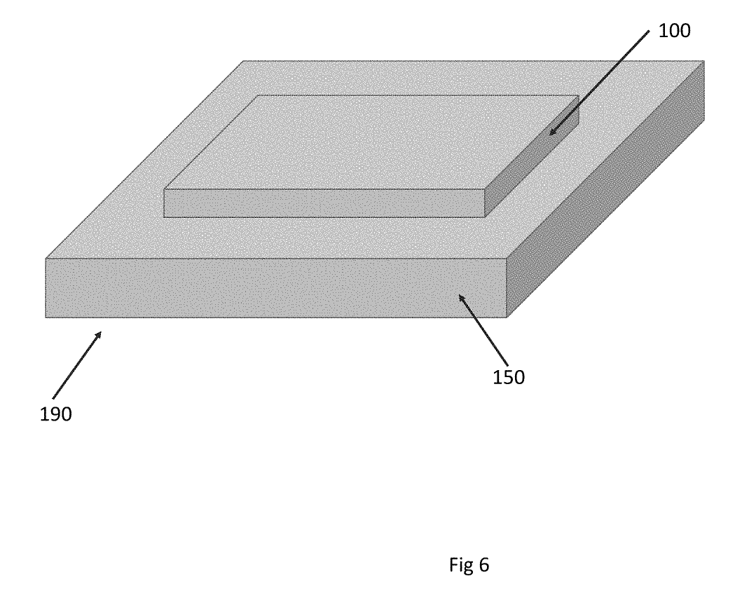

### DETAILED DESCRIPTION OF THE INVENTION

Reference will now be made in greater detail to a preferred embodiment of the invention, an example of which is illustrated in the accompanying drawings. Wherever possible, the same reference numerals will be used throughout the drawings and the description to refer to the same or like parts.

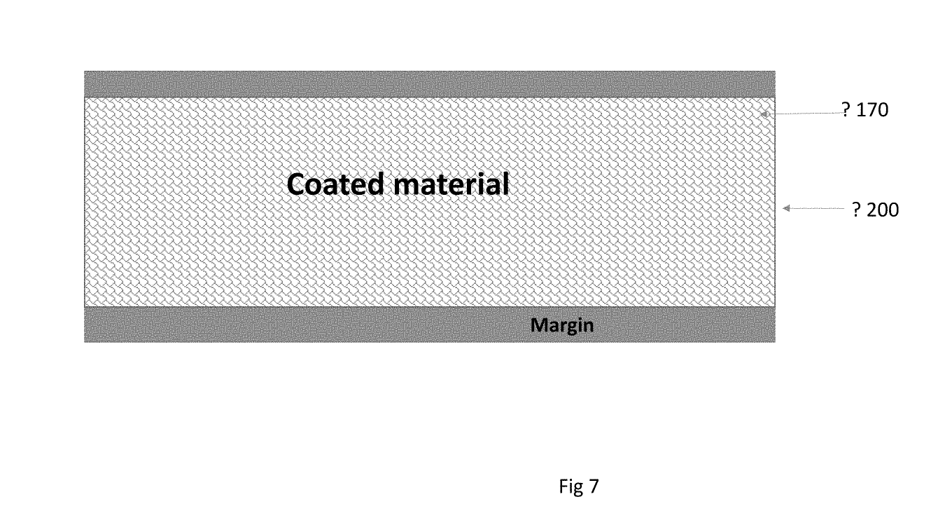

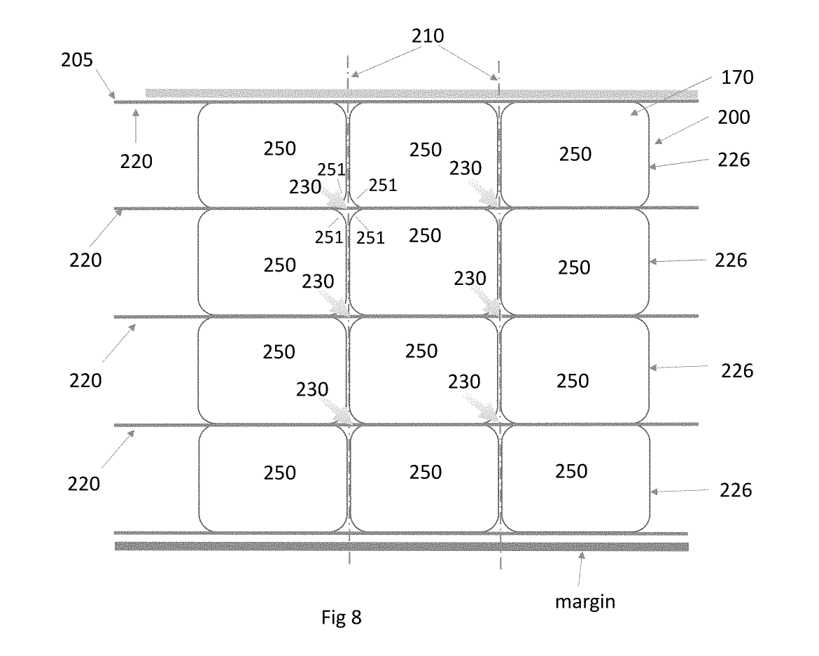

The method for producing transdermal devices of the present invention includes providing layered web material 170 as shown in FIG. 7. Starting web 200 of layered web material 170 is transported to cutting station 205 where it is through-cut along horizontal lines 220 into separable strips 226, and kiss-cut along vertical lines 210 down to the depth of the release liner as shown in FIG. 8. Portions 230 where horizontal lines 220 and vertical lines 210 intersect can be cut out to provide rounded corners for zones 250 of layered web material 170. For example, dies can be used to kiss-cut or punch out portions 230. Portions 230 can have a star or diamond-shaped so as to provide rounded corners 251 for zones 250. Rounded corners 251 can be cut before or after horizontal lines 220 are made for horizontal through-cuts down the length of the web that form the separable strips.

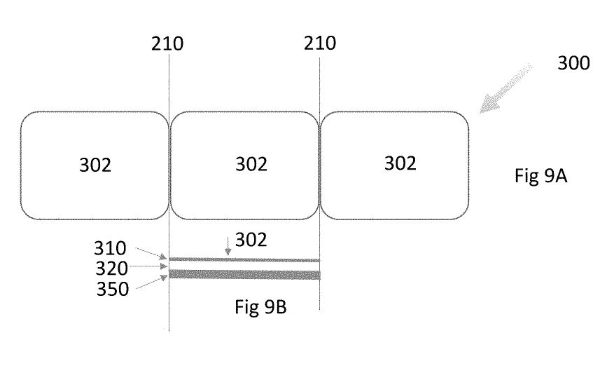

Starting web 200 can be cut into one or more separate webs 300, each made up of a series of adjoining segments 302 that are delimited by vertical lines 210 which can be kiss-cut as shown in FIG. 9A. Each of segments 302 will each include backing film layer 310, at least one drug-in-adhesive layer 320, and strippable release layer 350 as shown in FIG. 9B.

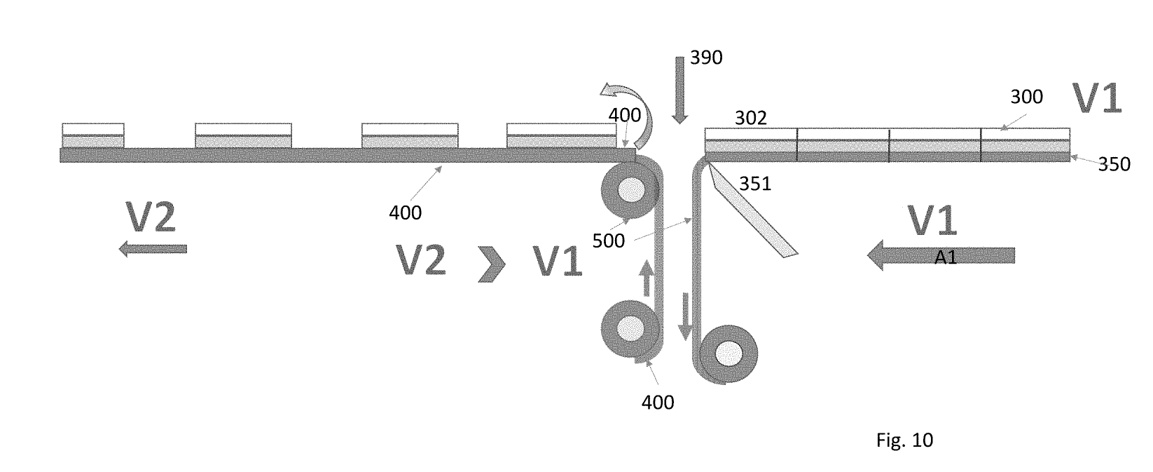

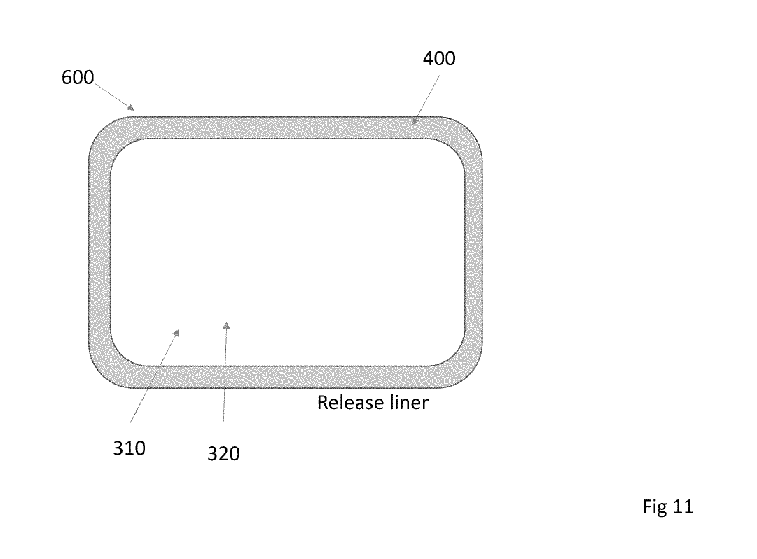

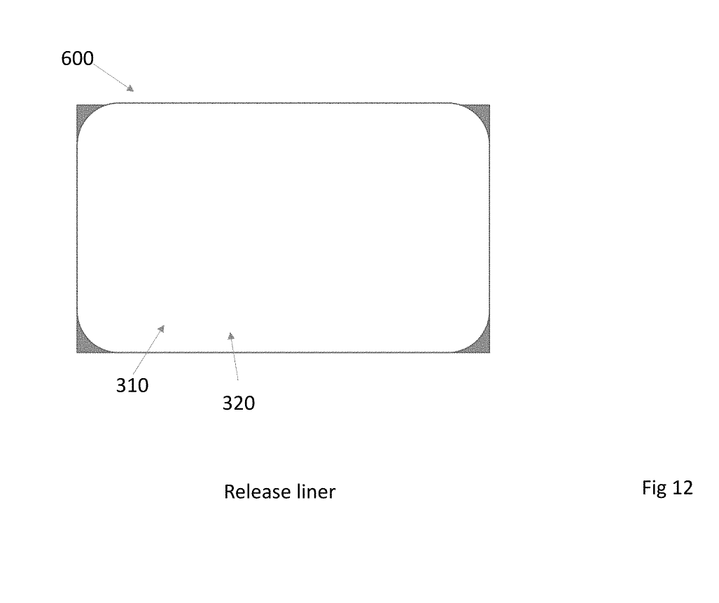

As shown in FIG. 10, web 300 is moved along at a first average velocity (V1) in the direction of arrow A1 to transfer station 390 where web 300 faces web of release liner material 400. Web of release liner material 400 moves at a second average velocity (V2). Second average velocity (V2) is greater than first average velocity (V1). Transfer machinery 500 detaches segments 302 from release layer 350 and affixes the detached segments 302 to release liner material 400 which is faster moving. Transfer machinery 500 can accomplish the transfer using a sharp edge on the machinery that raises the front rim 351 of the forward moving segment 302. Front rim 351 then contacts web of release liner 400 that moves faster than web 300. Front rim 351 then adheres to web of release liner 400 resulting in transfer of segment 302 to web of release liner 400. Web of release liner 400 is sufficiently wider to provide a broader base in the direction orthogonal to its direction of transfer, and its faster movement with respect to web 300 results in a desired level of lateral spacing, so that web of release liner 400 can then be cut to form patch 600 as shown in FIG. 11. Patch 600 can be a transdermal patch including film layer 310, at least one drug-in-adhesive layer 320 and release liner 400. Alternatively, web of release liner 400 can be the same width as patch 600, yielding a finished product with no extension of release liner 400 at the sides of patch 600 as shown in FIG. 12.

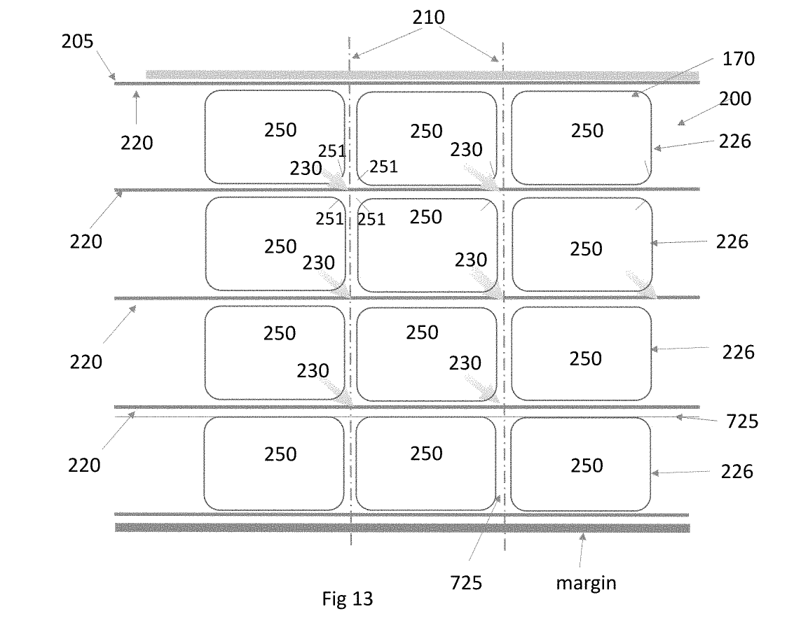

In some cases, the vertical kiss-cut 210 does not result in a clean separation of the contiguous patches, because the adjacent adhesive surfaces along the kiss-cut re-adhere after the kiss-cut. FIG. 13 illustrates an embodiment in which horizontal lines 220 and vertical lines 210 include a pair of horizontal lines 720 and vertical lines 210 leaving a thin strip of waste material 725 between the patches. Thin strip 725 is removed with the rounded corners leaving a narrow space between contiguous patches 700, preventing re-adherence between the contiguous patches 700.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

What is claimed is:

1. A method for manufacturing a transdermal device comprising the steps of:
providing a first web of material, the first web of material including a film layer, at least one drug-in-adhesive layer and a first release liner;
cutting a plurality of vertical lines and horizontal lines in the first web of material to delimit a plurality of segments of the first web of material;
peeling away portions of the first release liner from the plurality of segments of the first web material; and
transferring the plurality of segments of the first web material beneath the peeled away portions of the first release liner to a second web of a release liner,
wherein the transferred plurality of segments each comprise the transdermal device.

2. The method of claim 1 wherein the transdermal device is a transdermal patch.

3. The method of claim 1 wherein the horizontal lines are through-cut providing separable strips of the first web of material.

4. The method of claim 1 wherein the vertical lines are kiss-cut through the film layer and the at least one drug-in-adhesive layer down to a depth of the first release liner.

5. The method of claim 1 further comprising the step of:
cutting out areas of the first web of material where the horizontal and vertical line intersect to provide rounded corners for the plurality of segments of the first web material.

6. The method of claim 5 wherein the step of cutting out areas of the first web is performed by stamping.

7. The method of claim 5 wherein the step of cutting out areas of the first web is performed as a kiss-cut.

8. The method of claim 1 wherein the first web of material is moved along at a first average velocity (V1) to a transfer station where the first web of material faces the second web of the release liner material and the second web of the release liner material moves at a second average velocity (V2), the second average velocity (V2) is greater than first average velocity (V1) to provide a predetermined spacing of the transferred plurality of segments on the second web of the release layer.

9. The method of claim 8 further comprising the step of cutting the transferred plurality of segments apart.

10. The method of claim 1 wherein a front rim of the first web of material contacts the second web of the release liner and the front rim then adheres to the second web of the release liner resulting in transfer of the segment of the first web material beneath the peeled away portion of the first release liner to the second web of the release liner.

11. The method of claim 10 wherein the second web of the release liner is wider than the width of the transferred plurality of segments.

12. The method of claim 10 wherein the second web of the release liner has a width which is substantially the same as the width of the transferred plurality of segments.

13. The method of claim 1 wherein each of the plurality of vertical lines and the plurality of horizontal lines are a pair of lines.

14. A transdermal device produced by the method of claim 1.

15. The transdermal device of claim 14 wherein the transdermal device is a transdermal patch.

16. The transdermal device of claim 14 wherein the horizontal lines are through-cut providing separable strips of the first web of material and the vertical lines are kiss-cut through the film layer and the at least one drug-in-adhesive layer down to a depth of the first release liner.

17. The transdermal device of claim 14 wherein the method further comprises the step of:
cutting out areas of the first web of material where the horizontal and vertical line intersect to provide rounded corners for the plurality of segments of the first web material.

18. The transdermal device of claim 14 wherein the first web of material is moved along at a first average velocity (V1) to a transfer station where the first web of material faces second web of the release liner material and the second web of the release liner material moves at a second average velocity (V2), the second average velocity (V2) is greater than first average velocity (V2) to provide a predetermined spacing of the transferred plurality of segments on the second web of the release layer.

19. The transdermal device of claim 14 wherein the second web of the release liner is wider than the width of the transferred plurality of segments.

20. The transdermal device of claim 14 wherein the second web of the release liner has a width which is substantially the same as the width of the transferred plurality of segments.

21. The method of claim 5, wherein the horizontal lines are through-cut providing separable strips of the first web of material, the vertical lines are kiss-cut through the film layer and the at least one drug-in-adhesive layer down to a depth of the first release liner, the first web of material is moved along at a first average velocity (V1) to a transfer station where the first web of material faces the second web of the release liner material and the second web of the release liner material moves at a second average velocity (V2), the second average velocity (V2) is greater than first average velocity (V1) and wherein the step of cutting out areas of the first web further comprises cutting a piece of the first web down to a depth of the first release liner of a previous kiss-cut and through-cut direction to provide a strip between adjacent transdermal devices and removing the strip leaving between adjacent transdermal devices a space to facilitate the removal of the transdermal device and transfer from the first web (V1) moving at first web velocity to the second web moving at the second web velocity (V2).
